(19) United States
(12) Patent Application Publication
(10) Pub. No.: US 2025/0022595 A1
(43) Pub. Date: Jan. 16, 2025

(54) AI RECOMMENDATION ARCHITECTURE FOR MEDICAL PRESCRIPTIONS

(71) Applicant: Oracle International Corporation, Redwood Shores, CA (US)

(72) Inventors: Amir Hossein Rezaeian, San Mateo, CA (US); Pranav Varia, Bothell, WA (US)

(21) Appl. No.: 18/222,392

(22) Filed: Jul. 14, 2023

(51) Int. Cl. G16H 50/20; G06F 40/40; G16H 10/60; G16H 20/10; G16H 50/30

(52) U.S. Cl. CPC G16H 50/20; G06F 40/40; G16H 10/60; G16H 20/10; G16H 50/30

(57) ABSTRACT

Techniques for selecting medical items for presentation using an artificial intelligence architecture are provided. In one technique, summary note data that is composed by a physician for a patient is received. A machine-learned (ML) language model generates, based on the summary note data, a set of feature values. A profile of the patient and a profile of the physician are identified. An ML recommendation model determines, based on the profile of the patient, the profile of the physician, and the set of feature values, a plurality of candidate medical items. An ML reinforcement learning model generates a ranking of the plurality of candidate medical items. A subset of the plurality of candidate medical items is caused to be presented on a screen of a computing device based on the ranking.

### TECHNICAL FIELD

The present disclosure relates to artificial intelligence (AI) and, more specifically, to an AI architecture for identifying and selecting medical items.

### BACKGROUND

Computer technology has helped advance many industries, including the medical industry. However, there are many touchpoints between a physician and patient that require the physician to make important, potentially life-changing, decisions based primarily on the physician's own limited knowledge of that patient and experience with that patient and other patients.

For example, after examining a patient, a physician typically composes a summary note detailing the patient's health issue, potential recommendations, and follow-up. This information may be saved in a secure electronic health record (EHR) database. Then, given a diagnosed health problem, the physician prescribes, to the patient, medicine or tests from a list of medical items.

As a specific example, if the physician desires to order a chest X-ray for the patient, the physician enters “chest x-ray” into a search field of a user interface, causing over forty options to be displayed in alphabetical order, each option corresponding to a different type of chest x-ray that may be prescribed. One problem is that alphabetic order does not necessarily mean relevant order. The most relevant and correct medical items should be displayed at the top of the list. Displaying the most appropriate medical items to prescribe will assist physicians in prescribing the most appropriate treatment for a patient, which will reduce medical costs, reduce time to medical resolution, increase quality of life measures for patients, and potentially increase lifespans.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

In the drawings:

FIG. 1 is a block diagram that depicts an example AI medical item selection system, in an embodiment;

FIG. 2 is a flow diagram that depicts an example process for selecting candidate medical item, in an embodiment;

FIG. 3 is a block diagram that illustrates a computer system upon which an embodiment of the invention may be implemented;

FIG. 4 is a block diagram of a basic software system that may be employed for controlling the operation of the computer system.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

### General Overview

A system and method for recommending medical prescriptions using an AI recommendation architecture are provided. The AI recommendation architecture comprises multiple components, including a large language model (LLM), a recommendation model, and a reinforcement learning from human feedback (RLHF) model. The LLM and RLHF models enhance the recommendation model. The LLM model extracts relevant features from a physician's summary note in an unstructured form and stores the extracted features as structured data, which can be utilized for training the recommendation model and at inference time. The RLHF model generates a ranking of a list of recommended medical items that the recommendation model outputs.

Embodiments improve computer-related technology pertaining to selecting medical items for presentation. Traditional item selection models are not capable of incorporating a large text corpus into modeling primarily due to significant latency at training and inference, and also due to scalability. Embodiments can still leverage a LLM to convert unstructured summary note data into structured data that a machine-learned (ML) recommendation model can consume.

### Machine-Learning

Machine learning is the study and construction of algorithms that can learn from, and make predictions on, data. Such algorithms operate by building a model from inputs in order to make data-driven predictions or decisions. Thus, a machine learning technique is used to generate a statistical model that is trained based on a history of attribute values. The statistical model is trained based on multiple attributes (or factors) described herein. In machine learning parlance, such attributes are referred to as “features.” To generate and train a statistical prediction model, a set of features is specified and a set of training data is identified.

Embodiments are not limited to any particular machine learning technique for training different models as described herein. Example machine learning techniques include linear regression, logistic regression, random forests, naive Bayes, and Support Vector Machines (SVMs). Some machine learning techniques may be supervised while others may be unsupervised. Advantages that machine-learned models have over rule-based models include the ability of machine-learned models to output a probability (as opposed to a number that might not be translatable to a probability), the ability of machine-learned prediction models to capture non-linear correlations between features, and the reduction in bias in determining weights for different features.

A machine-learned prediction model may output different types of data or values, depending on the input features and the training data. For example, training data may comprise, for each training instance, multiple feature values, each corresponding to a different feature. In order to generate the training data, information about each of multiple past prescriptions is analyzed to compute the different feature values. In this example, the label (or dependent variable) of each training instance may be whether the corresponding physician selected a corresponding medical item for prescribing to a particular patient.

The term “physician” includes others in a clinical role that prescribe medication and treat patients, such as nurse practitioners, physician assistants, etc., all of which may be referred to herein as “physicians” or “clinicians.”

### System Overview

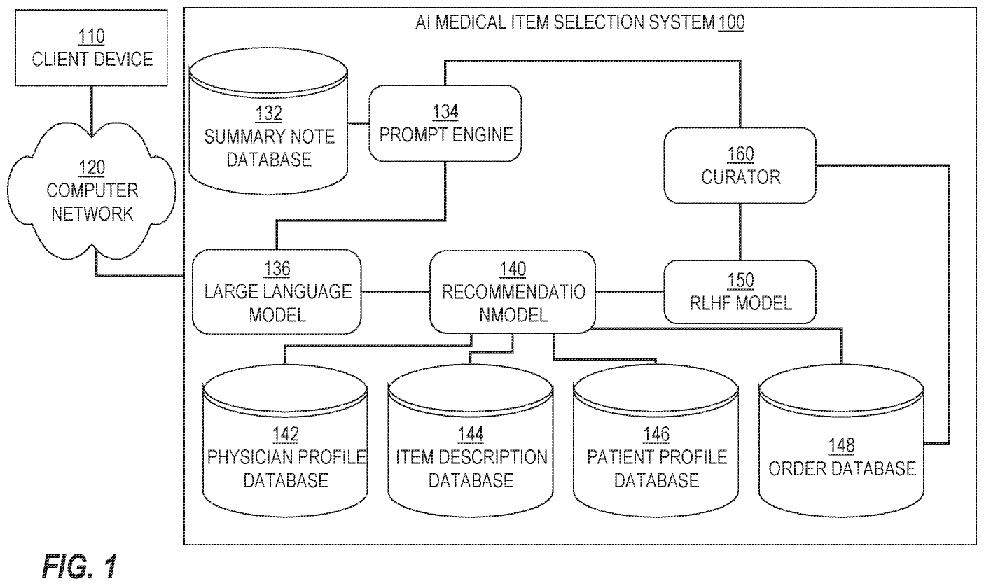

FIG. 1 is a block diagram that depicts an example AI medical item selection system 130, in an embodiment. Client device 110 is communicatively coupled to AI medical item selection system 130 over a computer network 120. Computer network 120 broadly represents a one or more local area networks (LANs), wide area networks (WANs), metropolitan area networks (MANs), or global interconnected internetworks (such as the public internet), or a combination thereof. Each such network may use or execute stored programs that implement internetworking protocols according to standards such as the Open Systems Interconnect (OSI) multi-layer networking model, including but not limited to Transmission Control Protocol (TCP) or User Datagram Protocol (UDP), Internet Protocol (IP), Hypertext Transfer Protocol (HTTP), and so forth. All components described herein may be configured to connect to the computer network 120. The various components depicted in FIG. 1 may also communicate with each other via direct communications links that are not depicted in FIG. 1.

Client device 110 may be a desktop computer, a laptop computer, a tablet computer, a watch or other wearable device, or any other portable computing device. A physician operates client device 110 by composing notes during or after a patient visit. Thus, client device 110 may be physically located in a physician's office.

“Composing notes” may involve the physician (or an assistant) making selections on a screen from a menu via a cursor control device (or “mouse”), selecting keys on a physical keyboard, selecting keys on a screen that displays a soft, or digital, keyboard, or orally dictating a summary. In the latter scenario, software on client device 110 (or remote thereto) performs audio processing on the oral dictation, converting an audio signal to text. The converted text may be modified automatically based on a medical dictionary and/or modified manually by the physician or assistant. The digital summary note is stored in an electronic record or account (not depicted) of the patient. The summary note become a part of the patient's history.

The summary note may be automatically transmitted to AI medical item selection system 130 upon completion of the summary note. For example, the physician selects a “Done” button, in a user interface, that is adjacent to a text field through which the physician enters the summary note, indicating that the physician no longer has any more notes to enter. Software executing on client device 110 causes summary note to be stored persistently (e.g., in a remote storage system) and causes the summary note to be transmitted to AI medical item selection system 130. As another example, after the lapse of a period of time (e.g., ten seconds) of no activity, client device 110 automatically transmits the summary note to AI medical item selection system 130. Additionally or alternatively, the physician may provide input that selects a summary note (or a set of summary notes) and a destination of the summary note (such as an icon representing AI medical item selection system 130 or a location node of AI medical item selection system 130).

### AI Medical Item Selection System

AI medical item selection system 130 comprises a summary note database 132, a prompt engine 134, a large language model (LLM) 136, a recommendation model 140, a physician profile database 142, an item description database 144, a patient profile database 146, an order database 148, a reinforcement learning from human feedback (RLHF) model 150, and a curator 160. Prompt engine 134, LLM 136, recommendation model 140, RLHF model 150, and curator 160 are implemented in software, hardware, or any combination of software and hardware.

Summary note database 132 stores a set of zero or more summary notes for each patient of multiple patients. Each summary note may be associated with (a) a timestamp of when the summary note was created and/or saved persistently and/or (b) a name and/or identifier of the physician that composed the summary note. Each summary note comprises a string of unstructured text of medical-related information pertaining to a patient. Each summary note may comprise one or more of the following types of information: potential diagnoses, one or more risk factors, and potential treatments, such as medicine, tests, and/or physical therapy. Additionally, a summary note may include information about the patient's medical history and/or a general impression of the physician.

### Prompt Engine

Prompt engine 134 receives a summary note, automatically generates a prompt, and sends the prompt and summary note to LLM 136. Prompt engine 134 receives a summary note when the summary note is created, when the summary note is stored in summary note database 132, when a user (e.g., a physician or administrator) provides input that selects the summary note, or as part of batch of summary notes that prompt engine 134 periodically receives and processes.

A prompt is a set of one or more instructions that LLM 136 will use to extract certain features from a summary note. For example, the set of one or more instructions may (1) identify features of a summary note to extract and (2) specify a format in which the extracted features should be stored or organized (e.g., a specific JSON (JavaScript Object Notation) format). Each prompt that prompt engine 134 sends to LLM 136 may be the same prompt, regardless of the summary note.

In an embodiment, prompt engine 134 generates a prompt that is tailored to a particular medical field or medical department. Physicians in different medical fields or departments may record different types of information. Examples of medical fields or departments include one medical department handling ocular matters and another medical department handling cardiac matters. Thus, prompt engine 134 may first identify a particular medical field or department and then select a set of instructions associated with that particular medical field/department. Identification of a particular medical field/department may be based on field data that is stored in a summary note. Additionally or alternatively, a source of a summary note may be associated with a particular medical field/department and prompt engine 134 identifies the source and then the medical field/department associated with that source.

In a related embodiment, AI medical item selection system 130 includes multiple prompt engines, each associated with a different medical field/department. Thus, each prompt engine is configured to generate a specific set of instructions that are tailored to its associated medical field/department. Nevertheless, each prompt engine is able to send those instructions to LLM 136.

### Large Language Model

LLM 136 is a generative language model that takes what it has learned from the training examples it has been shown and creates something entirely new based on those training examples. LLMs are one type of generative AI since they generate novel combinations of text in the form of natural-sounding language. The term “generative” emphasizes the content-creating function of these models.

LLM 136 receives a prompt and a summary note from prompt engine 134 and converts the unstructured text (e.g., a string of characters) of the summary note into a structured data format, such as a JSON file. Training LLM 136 may be performed in three stages: a general language training phase, a specific language training phase, and a specific health provider training phase. In the general training phase, LLM 136 is trained based on training data that is not specific to a particular industry or field of endeavor. An example of such training data is Wikipedia articles. In the specific training phase, parameters of LLM 136 are tune by re-training LLM 136 only on training data that is specific to a particular industry or field of endeavor. An example of such training data is all available medical and biomedical science literature.

In the specific health provider training phase, LLM 136 is further tuned by re-training based on a corpus of data that is specific to a given health provider, including electronic health records (EHR) of patients. The separating training of LLM 136 in this fashion increases the robustness of the model and decreases the latency of training. The first two training stages are agnostic to the healthcare provider and, therefore, the same pre-trained model is built once for all healthcare providers. For re-training at the third stage, the EHR data cannot be directly used for training due to HIPPA and other regulations. Nevertheless, the EHR data may be anonymized by applying differential privacy techniques.

After training, given a prompt and a summary note, LLM 136 compares the summary note with all existing medical data upon which the model was trained and then converts, according to the prompt, the summary note into structured features, such as potential diagnoses features, risk factor features, and potential treatment features. Thus, the output of LLM 136 is in a structured data format and is sent to recommendation model 140. Some of the data in the output may include data not only from the summary note, but data upon which LLM 136 was trained, which may include other summary notes in summary note database 132.

In an embodiment, prompt engine 134 (or another component of system 130) analyzes output generated by LLM 136 before the output is transmitted to recommendation model 140 as input. For example, prompt engine 134 verifies whether the output is in the structured data format and/or the type of data is correct. If not, then prompt engine 134 may send additional instructions to LLM 136 with the original summary note until LLM 136 produces output that conforms to the expected structured data format and contains the correct type of data. For example, prompt engine 134 determines whether the feature names are correct and whether the feature values are the correct data type. For example, if a feature value for a particular feature is supposed to be a number, but the feature value outputted by LLM 136 contains non-numeric characters, then prompt engine 134 ensures that the output from LLM 136 is not sent to recommendation model 140.

### Recommendation Model

Recommendation model 140 is a machine-learned model that takes input from multiple sources, including structured data outputted from LLM 136. Example sources for generating training data and composing inference data include physician profile database 142, item description database 144, patient profile database 146, and order database 148. Physician profile database 142 stores a profile for each physician of multiple physicians, which include physicians who have composed summary notes that are stored in summary note database 132. A physician profile might include a physician identifier (that uniquely identifies a physician from other physicians indicated in physician profile database 142), an age of the physician, one or more areas of practice in which the physician is engaged in medical practice, a seniority level of the physician in each area of practice, a list of academic degrees and credentials of the physician, a list of specialties and areas of expertise of the physician, a list of professional memberships of which the physician is a part, an availability and a practice location of the physician, an average rating of the physician received from patients, a list of languages spoken by the physician, and a list of clinical interests that the physician has.

Item description database 144 stores a description of each medical item that may be prescribed to a patient and, optionally, has been prescribed in the past, as indicated in order database 148. The description comprises a set of values of a set of pre-defined features. It is possible that for some item descriptions, some pre-defined features will not have any values. In such a case, as a pre-processing step of a ML pipeline, those features with no values are imputed using the statistical properties of the data.

Patient profile database 146 stores a profile for each patient of multiple patients. A patient profile may include an age of the patient, a geographic location of the patient (e.g., a country, a state, or a home address), a gender or sex of the patient, and medical history of the patient, which medical history may include one or more past diagnoses for the patient, medications prescribed to the patient, treatment plans prescribed to the patient, dates of immunizations that the patient received, allergies of the patient, laboratory tests received by the patient, results of those laboratory tests, and one or more past surgeries that the patient has received (e.g., a type of surgery and when the surgery occurred).

Order database 148 stores data about previous orders for medical items, such as medicine, tests, or other treatments. Each entry in order database 148 corresponds to a different order. Each entry includes a physician identifier of a physician that prescribed the medical item of the corresponding order (which physician identifier identifies a profile in physician database 142), a patient identifier of a patient that was prescribed the medical item of the corresponding order (which patient identifier identifies a profile in patient database 146), and a medical item identifier of the medical item that was prescribed (which medical item identifier identifies an entry in item description database 144). Each order entry may also include a timestamp that may be used to identify a temporal ordered set of orders for a particular patient. The temporal order of a patient's past prescriptions/orders may be used to train recommendation model 140 and as input at inference time.

At least some physicians identified in physician profile database 142 are also identified in order database 148; however, some physicians identified in physician profile database 142 might not be identified in order database 148. Similarly, at least some items identified in item description database 144 are also identified in order database 148; however, some items identified in item description database 144 might not be identified in order database 148. Similarly, at least some patients identified in patient profile database 146 are also identified in order database 148; however, some patients identified in patient profile database 146 might not be identified in order database 148.

For a particular summary note, in addition to the features extracted by LLM 136, the other input data to recommendation model 140 include a physician profile from physician profile database 142 (of the physician that composed or authored the summary note), one or more entries from item description database 144 (if the patient has been prescribed one or more medical items in the past), a patient profile from patient profile database 146, and one or more orders from order database 148 (if the patient has been prescribed one or more medical items in the past).

Recommendation model 140 outputs a list of one or more relevant candidate medical items for prescription to a patient, where the candidates are personalized based the patient profile (that was input to recommendation model 140), the physician profile (that was input to recommendation model 140), and item description data (that was input to recommendation model 140) and medical item identifiers of one or more medical items that have been previously prescribed to the patient (according to order database 148). If there are multiple candidate medical items in the outputted list, then the candidate medical items may be ordered based on a relevance score that recommendation model 140 assigns to the candidate medical item.

A list comprises a reference to each candidate medical item indicated in the list. A reference may comprise a name of the candidate medical item and/or a description of the candidate medical item. Different types of medical items may have different sets of descriptions. For example, a reference to a particular medicine may have a name of the medicine's manufacturer/source, an expiration date of the medicine, and a typical recommended dosage. A reference to a test or exam may have a type of test/exam (e.g., blood test v. x-ray v. MRI), a part of the body that is being tested/examined, a location of a provider of the test/exam, a length of time to administer the test/exam, and one or more actions for the patient to perform prior to taking the test/exam (e.g., fasting 24 hours before the test, refraining from exercise for an hour before the test).

In an embodiment, recommendation model 140 comprises a neural network that comprises embedding layers and two or more fully connected layers. The embedding layers are hidden layers of the neural network and generates (or outputs) the embeddings (or vectors) that are latent representations of the input data. Specifically, at some point in the embedding layers, there is a layer (or layer portion) that produces a patient-specific embedding that is based only on input related to the patient, another layer (or layer portion) that produces a physician-specific embedding that is based only on input related to the physician, and another layer (or layer portion) that produces a medical item-specific embedding for each medical item that has been prescribed to the patient in the past. Then, downstream from those specific embeddings, a portion of the embedding layers produces a single embedding that is based on the specific embeddings and represents all inputs.

In a related embodiment, recommendation model 140 is a self-attentive recommendation (SAR) model that comprises multi-head self-attention layers between the embedding layers and the fully connected layers. The self-attention layers take the embeddings (outputted from the embedding layers) as input, converts the embeddings into three matrices through linear projections. The matrices include a queries matrix, a keys matrix, and a values matrix. These three matrices are constructed by a dot product with the embedding vectors. The three matrices are fed into each attention layer. The self-attention layers are unordered and are based on transformer units. Thus, the computation in the SAR model can be parallelized, reducing the latency both during the training phase and at runtime or inference time.

In an embodiment, the position of sequence and/or temporal information associated with prior orders for a particular patient (as identified by analyzing order database 148 based on patient identifier) is incorporated via a learnable position embedding that is added to the embeddings vectors constructed from the corresponding data for the patient in question. Therefore, recommendation model 140 also learns the temporal correlations of relevant medical items. In this way, recommendation model 140 learns the sequence of medical items that are associated with each other and should be prescribed within a time frame. For example, recommendation model 140 may learn that test B typically comes after a prescription for medication A for most patients that are similar to the patient in question (or for the physician in question) and since the patient in question has been (or the physician in question has) prescribed medication A, then recommendation model 140 identifies test B as a candidate item to include in a list of candidate items.

In an embodiment, recommendation model 140 is trained for and used by a single medical organization or facility. In this way, information about all prescribable medical items that are known to a medical organization or facility is used to train recommendation model 140. Therefore, recommendation model 140 is not trained based on medical items that are unknown to the medical organization or facility. Nevertheless, if a new medical item is added to the item description database 144, recommendation model 140 is still able to generate a list of relevant candidate medical items since some of the features of the new medical item may be known and either the physician or patient (or both) might be known.

Training data for recommendation model 140 is automatically composed by individually generating multiple individual training instances. A training instance comprises input feature data and label data. The input feature data includes data outputted by LLM 136 (and based on a summary note, which originates from summary note database 132), a physician profile, a patient profile, and order history of the patient (if such order history exists), where the order history comprises zero or more item descriptions, each corresponding to a different medical item that was prescribed to the patient before the time the summary note was authored. Label data for a training instance comprises a medical item identifier that was prescribed given the scenario corresponding to the data in the input feature data. Therefore, for each prescription indicated in order database 148, a different training instance may be generated. Also, if a patient has been prescribed multiple medical items in the past, then some of those medical items may appear in the order history portion of multiple training instances. For example, a patient has been prescribed (by the same or different physicians) medical items A, B, C, and D. A first training instance that has medical item D identified as label data, may include item descriptions for medical items A, B, and C in the order history portion of the first training instance; whereas a second training instance that has medical item C identified as label data, may include item descriptions for medical items A and B in the order history portion of the second training instance.

In some scenarios, order database 148 is not available or is very limited with only a few entries. Such a situation is referred to as a cold-start problem. However, embodiments are able to overcome the cold-start problem to a certain extent since the summary notes are available. The summary notes contain prescription-related information in the form of potential treatments and the output data from LLM 136 corresponding to potential treatments may be used as label data. In this way, recommendation model 140 can still be trained and used in a cold-start scenario.

### Reinforcement Learning and Curator

As described above, recommendation model 140 outputs a list of candidate medical items, which may be presented, such as on a screen of a computing device associated with the physician, such as client device 110.

Alternatively, in an embodiment, RLHF model 150 adjusts or re-ranks the list of candidate medical items and causes the re-ranked list to be presented on the screen of the computing device associated with the physician. At a high-level, RLHF is an efficient way of maximizing click-rate with a human-in-loop. This RLHF stage may be considered a fine-tuning stage of recommendation model 140. This separation helps in terms of stability and scalability, and also effectively takes into account the human-in-loop effect.

Medical items indicated in the initial list (from recommendation model 140) have relevance scores, which are used in a reward function of RLHF model 150. If the ranking from recommendation model 140 is correct for a candidate medical item and no intervention by curator 160 occurs, then RLHF model 150 is already optimized. Each time curator 160 intervenes in re-ranking, an RLHF agent (not depicted) penalizes the reward (which was initially computed by the relevance score) of RLHF model 150, and the RLHF agent attempts to maximize the reward by learning its past experiences. A penalty may be computed by subtracting a negative score from the reward function. The value of the negative score is a hyper-parameter. The reward may be maximized in multiple ways, such as by a stochastic gradient decent approach combined with techniques in the RL approach to find a trade-off between exploration and exploitation.

Training data for RLHF model 150 comprises data from order database 148. Each training instance in the training data comprises input feature data and label data. Input feature data of a training instance comprises a set of scores for a list of candidate medical items while label data of the training instance comprises a candidate medical item that a physician selected. The cold-start problem can be dealt with at the stage corresponding to recommendation model 140. The context of items and users is incorporated in recommendation model 140 and, thus, medical items can still be recommended even in the absence of order database 148.

In an embodiment, RLHF model 150 implements one or more constraints for the final ranking, boosts the score of fresher (or newer) content, and/or incorporates diversity and fairness. As an example of a constraint, RLHF model 150 implements a filtering rule that indicates that certain providers of medical items are to be removed from any list of candidate medical items. This filtering rule may be temporary. As an example of a score booster, if a candidate medical item is relatively new (e.g., became available to the public in the last six months), then a score of the candidate medical item is increased by 20%. Such percentage increase may be fixed throughout a certain period of time or may get smaller as time proceeds until the percentage increase is zero after the lapse of the certain period of time.

As an example of incorporating diversity, a diversity rule that RLHF model 150 implements is that at least four medical providers must be represented in a list of candidate medical items. As an example of incorporating fairness, a fairness rule that RLHF model 150 implements is that no more than 25% of candidate medical items in a list can be from a single medical provider. Such diversity and fairness rules may go into effect if the number of candidate medical items in a list is greater than a particular threshold number, such as ten.

In an embodiment, in response to a positive selection (by curator 160) of a reference to a candidate medical item in a re-ranked list, order database 148 is updated to include a new entry that identifies the physician in question, the patient in question, and the selected medical item. For example, the presented list of re-ranked candidates has metadata that includes a physician identifier, a patient identifier, and a medical item identifier for each candidate medical item in the re-ranked list. Curator 160's computer transmits, to order database 148 (or another device that is configured to communicate messages to order database 148), a payload that comprises the physician identifier, the patient identifier, and a medical identifier of the selected medical item.

Some generative language models suffer from hallucination, which is when a language model generates false information. Therefore, in an embodiment, curator 160 provides (e.g., through client device 110) one or more reasons why one or more candidate medical items (indicated in a re-ranked list from RLHF model 150) are not correct or should not have been suggested as candidates. The one or more reasons may be entered as text via a keyboard (whether physical or visual). Alternatively, a reason may be provided orally (e.g., by the physician), which audio is captured by a microphone and digitized, creating an audio file, and then the digital audio data in the audio file is converted to text data. The text data may be sent back to the prompt engine 134, which fine-tunes LLM 136, which reduces the likelihood of hallucination of LLM 136. Example reasons may be as follows: (1) “a pediatric patient and this medication is difficult for children to take because of the large pill”; and (2) “contraindicated given the patient has diabetes and heart disease” (i.e., multiple co-morbid conditions instead of a single one).

In an embodiment, curator 160 provides one or more reasons why one or more candidate medical items (indicated in a re-ranked list from RLHF model 150) are correct. Such reasons may be used to also update LLM 136.

In an embodiment, feedback from curator 160 is an update to a summary note that the physician composed and that triggered the generation of the list of candidate medical items in question. For example, the feedback may involve deleting a potential diagnosis and/or a potential treatment and adding a different potential diagnosis and/or a different potential treatment. In response to such feedback, the updated summary note is sent to prompt engine 134, which passes the updated summary note to LLM 136 along with a set of instructions and the process repeats, but with different output from LLM 136 based on the updated summary note, resulting in another list of candidate medical items that curator 160 views.

One or more candidate medical items that are indicated in a re-ranked list (and that were not removed based on input from curator 160), are presented (on a screen of a computing device, e.g., client device 110) to a physician, such as the physician who composed the summary note that triggered the generation of the re-ranked list. For example, the top N ranked candidate medical items are presented. As another example, all candidate medical items with a score above a particular score threshold are presented. As another example, the top N ranked candidate medical items whose scores are above the particular score threshold are presented.

Before and/or during the presentation of the one or more candidate medical items to the physician, the physician may also be presented with the order history of the patient. The order history (e.g., from order database 148) may be medical orders made by the physician and/or other physicians. This allows the physician to view the candidate medical items in context of what the patient has been prescribed in the past.

The physician provides input (whether orally, through a keyboard, or through touchscreen) that selects one or more candidate medical items or that ignores all presented candidate medical items.

### Process Overview

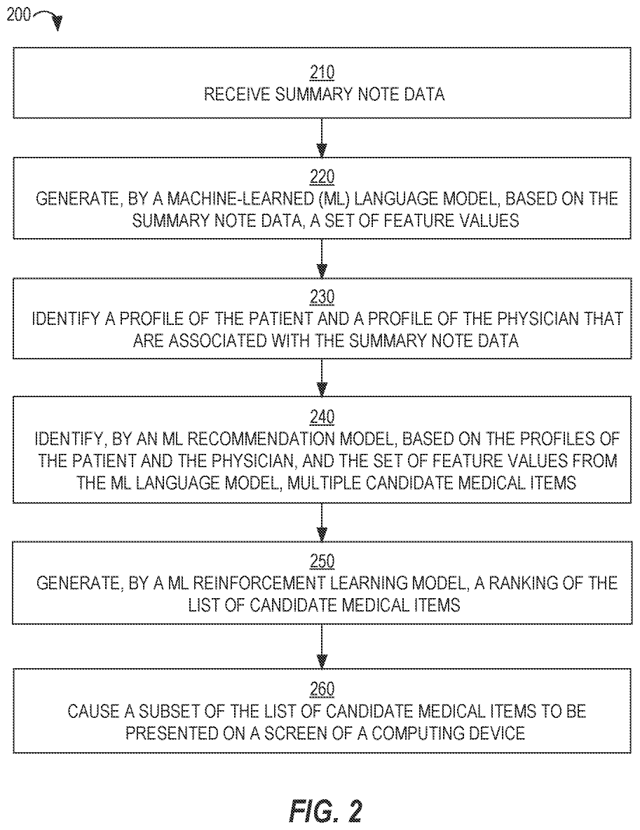

FIG. 2 is a flow diagram that depicts an example process 200 for selecting candidate medical item, in an embodiment. Process 200 may be performed by different components of system 130.

At block 210, summary note data that is composed by a physician for a patient is received. The summary note data may be originally composed on client device 110 and transmitted (e.g., automatically) to AI medical item selection engine 130 and stored in summary note database 132.

At block 220, an ML language model (e.g., LLM 136) generates a set of feature values based on the physician summary note data. The set of feature values correspond to a set of features that a prompt engine may have specified as prompt input (along with the summary note data) to the ML language model.

At block 230, a profile of the patient and a profile of the physician are identified. Block 230 may involve identifying a patient identifier and a physician identifier, both of which are associated with the summary note, and then using the two identifiers to look up respective profiles in databases 142 and 146. Block 230 may also involve using the patient identifier to identify entries in order database 148 that have the patient identifier. This last lookup may yield zero results if the patient is new or does not have a history of medical prescriptions.

At block 240, an ML recommendation model identifies a plurality of medical item candidates based on the profile of the patient, the profile of the physician, and the set of feature values from the ML language model. Block 240 involves inputting features from the two profiles into the ML recommendation model, which may comprise an embedding layer that generates an embedding for each input data item. Block 240 may also involve inputting data items corresponding to medical items that the patient in question has been prescribed previously. Such input may include temporal data that indicates a temporal order in which such medical items were prescribed to the patient.

At block 250, an ML reinforcement learning model (e.g., RLHF model 150) generates a ranking of the identified medical item candidates.

At block 260, a subset of the plurality of medical item candidates is caused to be presented on a screen of a computing device based on the ranking. The computing device may be the same computing device as client device 110. For example, if the number of candidate medical items is greater than a particular threshold number, then only the top N ranked candidate medical items are presented. If the number of candidate medical items is not greater than the particular threshold number, then all the identified candidate medical items may be presented. Block 260 may involve presenting higher ranked candidate medical items differently than lower ranked candidate medical items. For example, higher ranked candidate medical items may be presented at the top of a list of the candidate medical items. As another example, higher ranked candidate medical items may be highlighted differently than lower ranked candidate medical items, such as with bolded text, italicized text, underlined text, colored text, colored background, certain icons (e.g., a star image) that are presented adjacent to the highest ranked item(s), etc.

Process 200 may also involve receiving, from a curator, such as the physician in question, input that is relative to one or more candidate medical items in the subset. For example, the input may comprise a selection of one of the candidate medical items, indicating an intention by the physician to prescribe that medical item. This input may be used to update order database 148. Additionally or alternatively, the input may comprise text data that indicates one or more reasons why a particular candidate medical item should not have been recommended/presented. This data may be used to update the summary note and/or to update the ML language model.

### Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

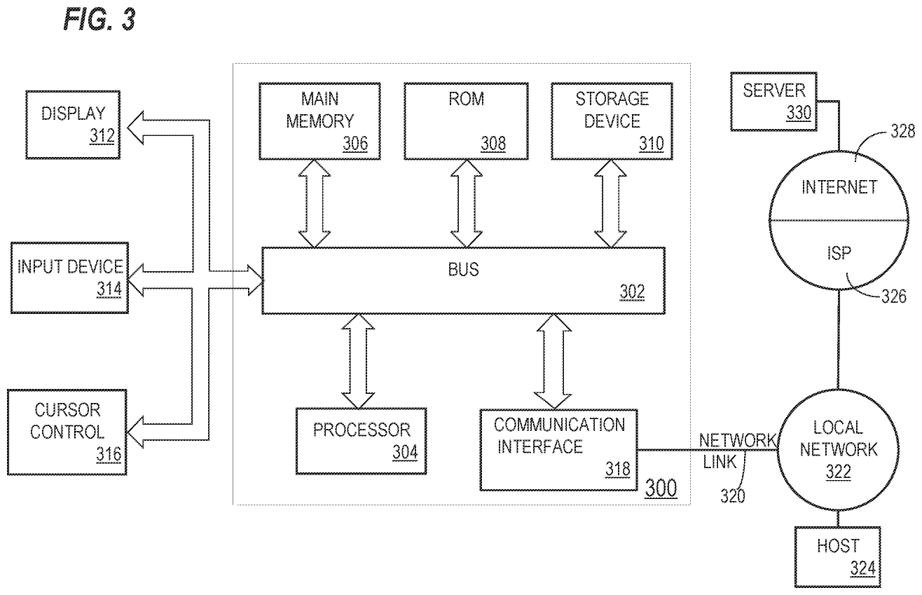

For example, FIG. 3 is a block diagram that illustrates a computer system 300 upon which an embodiment of the invention may be implemented. Computer system 300 includes a bus 302 or other communication mechanism for communicating information, and a hardware processor 304 coupled with bus 302 for processing information. Hardware processor 304 may be, for example, a general purpose microprocessor.

Computer system 300 also includes a main memory 306, such as a random access memory (RAM) or other dynamic storage device, coupled to bus 302 for storing information and instructions to be executed by processor 304. Main memory 306 also may be used for storing temporary variables or other intermediate information during execution of instructions to be executed by processor 304. Such instructions, when stored in non-transitory storage media accessible to processor 304, render computer system 300 into a special-purpose machine that is customized to perform the operations specified in the instructions.

Computer system 300 further includes a read only memory (ROM) 308 or other static storage device coupled to bus 302 for storing static information and instructions for processor 304. A storage device 310, such as a magnetic disk, optical disk, or solid-state drive is provided and coupled to bus 302 for storing information and instructions.

Computer system 300 may be coupled via bus 302 to a display 312, such as a cathode ray tube (CRT), for displaying information to a computer user. An input device 314, including alphanumeric and other keys, is coupled to bus 302 for communicating information and command selections to processor 304. Another type of user input device is cursor control 316, such as a mouse, a trackball, or cursor direction keys for communicating direction information and command selections to processor 304 and for controlling cursor movement on display 312. This input device typically has two degrees of freedom in two axes, a first axis (e.g., x) and a second axis (e.g., y), that allows the device to specify positions in a plane.

Computer system 300 may implement the techniques described herein using customized hard-wired logic, one or more ASICs or FPGAs, firmware and/or program logic which in combination with the computer system causes or programs computer system 300 to be a special-purpose machine. According to one embodiment, the techniques herein are performed by computer system 300 in response to processor 304 executing one or more sequences of one or more instructions contained in main memory 306. Such instructions may be read into main memory 306 from another storage medium, such as storage device 310. Execution of the sequences of instructions contained in main memory 306 causes processor 304 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage device 310. Volatile media includes dynamic memory, such as main memory 306. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 302. Transmission media can also take the form of acoustic or light waves, such as those generated during radio-wave and infra-red data communications.

Various forms of media may be involved in carrying one or more sequences of one or more instructions to processor 304 for execution. For example, the instructions may initially be carried on a magnetic disk or solid-state drive of a remote computer. The remote computer can load the instructions into its dynamic memory and send the instructions over a telephone line using a modem. A modem local to computer system 300 can receive the data on the telephone line and use an infra-red transmitter to convert the data to an infra-red signal. An infra-red detector can receive the data carried in the infra-red signal and appropriate circuitry can place the data on bus 302. Bus 302 carries the data to main memory 306, from which processor 304 retrieves and executes the instructions. The instructions received by main memory 306 may optionally be stored on storage device 310 either before or after execution by processor 304.

Computer system 300 also includes a communication interface 318 coupled to bus 302. Communication interface 318 provides a two-way data communication coupling to a network link 320 that is connected to a local network 322. For example, communication interface 318 may be an integrated services digital network (ISDN) card, cable modem, satellite modem, or a modem to provide a data communication connection to a corresponding type of telephone line. As another example, communication interface 318 may be a local area network (LAN) card to provide a data communication connection to a compatible LAN. Wireless links may also be implemented. In any such implementation, communication interface 318 sends and receives electrical, electromagnetic, or optical signals that carry digital data streams representing various types of information.

Network link 320 typically provides data communication through one or more networks to other data devices. For example, network link 320 may provide a connection through local network 322 to a host computer 324 or to data equipment operated by an Internet Service Provider (ISP) 326. ISP 326 in turn provides data communication services through the worldwide packet data communication network now commonly referred to as the “Internet” 328. Local network 322 and Internet 328 both use electrical, electromagnetic, or optical signals that carry digital data streams. The signals through the various networks and the signals on network link 320 and through communication interface 318, which carry the digital data to and from computer system 300, are example forms of transmission media.

Computer system 300 can send messages and receive data, including program code, through the network(s), network link 320 and communication interface 318. In the Internet example, a server 330 might transmit a requested code for an application program through Internet 328, ISP 326, local network 322 and communication interface 318.

The received code may be executed by processor 304 as it is received, and/or stored in storage device 310, or other non-volatile storage for later execution.

### Software Overview

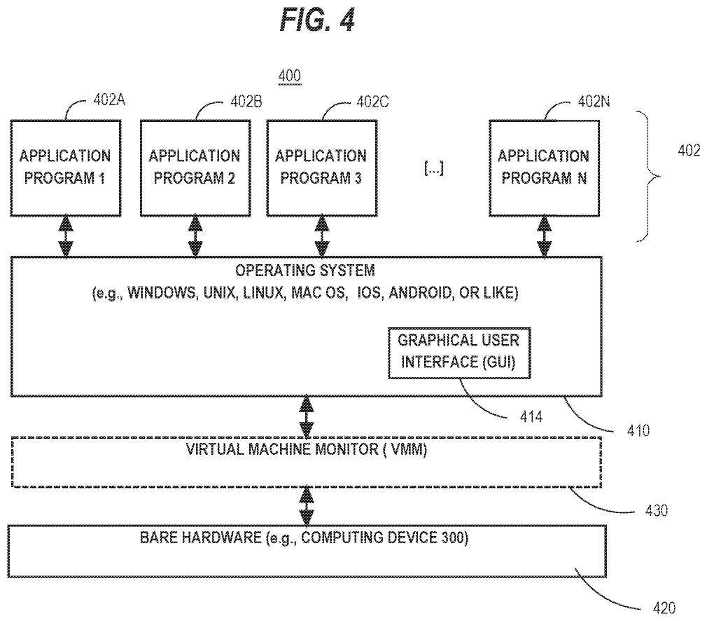

FIG. 4 is a block diagram of a basic software system 400 that may be employed for controlling the operation of computer system 300. Software system 400 and its components, including their connections, relationships, and functions, is meant to be exemplary only, and not meant to limit implementations of the example embodiment(s). Other software systems suitable for implementing the example embodiment(s) may have different components, including components with different connections, relationships, and functions.

Software system 400 is provided for directing the operation of computer system 300. Software system 400, which may be stored in system memory (RAM) 306 and on fixed storage (e.g., hard disk or flash memory) 310, includes a kernel or operating system (OS) 410.

The OS 410 manages low-level aspects of computer operation, including managing execution of processes, memory allocation, file input and output (I/O), and device I/O. One or more application programs, represented as 402A, 402B, 402C . . . 402N, may be “loaded” (e.g., transferred from fixed storage 310 into memory 306) for execution by the system 400. The applications or other software intended for use on computer system 300 may also be stored as a set of downloadable computer-executable instructions, for example, for downloading and installation from an Internet location (e.g., a Web server, an app store, or other online service).

Software system 400 includes a graphical user interface (GUI) 415, for receiving user commands and data in a graphical (e.g., “point-and-click” or “touch gesture”) fashion. These inputs, in turn, may be acted upon by the system 400 in accordance with instructions from operating system 410 and/or application(s) 402. The GUI 415 also serves to display the results of operation from the OS 410 and application(s) 402, whereupon the user may supply additional inputs or terminate the session (e.g., log off).

OS 410 can execute directly on the bare hardware 420 (e.g., processor(s) 304) of computer system 300. Alternatively, a hypervisor or virtual machine monitor (VMM) 430 may be interposed between the bare hardware 420 and the OS 410. In this configuration, VMM 430 acts as a software “cushion” or virtualization layer between the OS 410 and the bare hardware 420 of the computer system 300.

VMM 430 instantiates and runs one or more virtual machine instances (“guest machines”). Each guest machine comprises a “guest” operating system, such as OS 410, and one or more applications, such as application(s) 402, designed to execute on the guest operating system. The VMM 430 presents the guest operating systems with a virtual operating platform and manages the execution of the guest operating systems.

In some instances, the VMM 430 may allow a guest operating system to run as if it is running on the bare hardware 420 of computer system 300 directly. In these instances, the same version of the guest operating system configured to execute on the bare hardware 420 directly may also execute on VMM 430 without modification or reconfiguration. In other words, VMM 430 may provide full hardware and CPU virtualization to a guest operating system in some instances.

In other instances, a guest operating system may be specially designed or configured to execute on VMM 430 for efficiency. In these instances, the guest operating system is “aware” that it executes on a virtual machine monitor. In other words, VMM 430 may provide para-virtualization to a guest operating system in some instances.

A computer system process comprises an allotment of hardware processor time, and an allotment of memory (physical and/or virtual), the allotment of memory being for storing instructions executed by the hardware processor, for storing data generated by the hardware processor executing the instructions, and/or for storing the hardware processor state (e.g. content of registers) between allotments of the hardware processor time when the computer system process is not running. Computer system processes run under the control of an operating system, and may run under the control of other programs being executed on the computer system.

The above-described basic computer hardware and software is presented for purposes of illustrating the basic underlying computer components that may be employed for implementing the example embodiment(s). The example embodiment(s), however, are not necessarily limited to any particular computing environment or computing device configuration. Instead, the example embodiment(s) may be implemented in any type of system architecture or processing environment that one skilled in the art, in light of this disclosure, would understand as capable of supporting the features and functions of the example embodiment(s) presented herein.

### Cloud Computing

The term “cloud computing” is generally used herein to describe a computing model which enables on-demand access to a shared pool of computing resources, such as computer networks, servers, software applications, and services, and which allows for rapid provisioning and release of resources with minimal management effort or service provider interaction.

A cloud computing environment (sometimes referred to as a cloud environment, or a cloud) can be implemented in a variety of different ways to best suit different requirements. For example, in a public cloud environment, the underlying computing infrastructure is owned by an organization that makes its cloud services available to other organizations or to the general public. In contrast, a private cloud environment is generally intended solely for use by, or within, a single organization. A community cloud is intended to be shared by several organizations within a community; while a hybrid cloud comprises two or more types of cloud (e.g., private, community, or public) that are bound together by data and application portability.

Generally, a cloud computing model enables some of those responsibilities which previously may have been provided by an organization's own information technology department, to instead be delivered as service layers within a cloud environment, for use by consumers (either within or external to the organization, according to the cloud's public/private nature). Depending on the particular implementation, the precise definition of components or features provided by or within each cloud service layer can vary, but common examples include: Software as a Service (SaaS), in which consumers use software applications that are running upon a cloud infrastructure, while a SaaS provider manages or controls the underlying cloud infrastructure and applications. Platform as a Service (PaaS), in which consumers can use software programming languages and development tools supported by a PaaS provider to develop, deploy, and otherwise control their own applications, while the PaaS provider manages or controls other aspects of the cloud environment (i.e., everything below the run-time execution environment). Infrastructure as a Service (IaaS), in which consumers can deploy and run arbitrary software applications, and/or provision processing, storage, networks, and other fundamental computing resources, while an IaaS provider manages or controls the underlying physical cloud infrastructure (i.e., everything below the operating system layer). Database as a Service (DBaaS) in which consumers use a database server or Database Management System that is running upon a cloud infrastructure, while a DbaaS provider manages or controls the underlying cloud infrastructure, applications, and servers, including one or more database servers.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A method comprising:
receiving summary note data that is composed by a physician for a patient;
generating, by a machine-learned (ML) language model, based on the summary note data, a set of feature values;
identifying a profile of the patient and a profile of the physician;
determining, by an ML recommendation model, based on the profile of the patient, the profile of the physician, and the set of feature values, a plurality of candidate medical items;
generating, by an ML reinforcement learning model, a ranking of the plurality of candidate medical items;
causing a subset of the plurality of candidate medical items to be presented on a screen of a computing device based on the ranking;
wherein the method is performed by one or more computing devices.

2. The method of claim 1, wherein the set of feature values correspond to one or more of a potential diagnosis, a risk factor, or potential treatment.

3. The method of claim 1, wherein the plurality of candidate medical items include one or more of medicines, medical treatments, or medical tests.

4. The method of claim 1, further comprising:
identifying, based on a patient identifier of the patient, one or more medical items, each of which was previously prescribed to the patient;
wherein determining the plurality of candidate medical items by the ML recommendation model is also based on the one or more medical items.

5. The method of claim 4, further comprising:
receiving, from a user, input that selects a particular candidate medical item in the subset;
based on the input, updating an entry, in an order database, that corresponds to the patient to include a reference to the particular candidate medical item.

6. The method of claim 1, further comprising:
receiving, from a user, input that indicates that one or more candidate medical items in the subset are incorrect recommendations;
updating the ML reinforcement learning model based on the input.

7. The method of claim 1, further comprising:
receiving, from a user, input that indicates one or more reasons why one or more candidate medical items in the subset are incorrect recommendations;
updating the ML language model based on the input.

8. The method of claim 1, further comprising:
receiving, from a user, input that indicates one or more reasons why one or more candidate medical items in the subset are incorrect recommendations;
updating the summary note data based on the input to generate an updated summary note data;
generating, by the ML language model, based on the updated summary note data, a second set of feature values;
generating, by the ML recommendation model, based on the profile of the patient, the profile of the physician, and the second set of feature values, a second plurality of candidate medical items;
generating, by the ML reinforcement learning model, a particular ranking of the second plurality of candidate medical items;
causing a subset of the second plurality of candidate medical items to be presented on the screen of the computing device based on the particular ranking.

9. The method of claim 1, wherein:
receiving the summary note data is performed by a prompt engine;
the method further comprising:
generating a set of instructions, and
causing the ML language model to access the set of instructions and the summary note data;
generating the set of feature values comprises generating the set of feature values also based on the set of instructions.

10. The method of claim 9, further comprising:
prior to the ML recommendation model generating the plurality of candidate medical items, determining, by the prompt engine, whether the set of feature values satisfy one or more format criteria.

11. One or more non-transitory storage media storing instructions which, when executed by one or more computing devices, cause:
receiving summary note data that is composed by a physician for a patient;
generating, by a machine-learned (ML) language model, based on the summary note data, a set of feature values;
identifying a profile of the patient and a profile of the physician;
determining, by an ML recommendation model, based on the profile of the patient, the profile of the physician, and the set of feature values, a plurality of candidate medical items;
generating, by an ML reinforcement learning model, a ranking of the plurality of candidate medical items;
causing a subset of the plurality of candidate medical items to be presented on a screen of a computing device based on the ranking.

12. The one or more storage media of claim 11, wherein the set of feature values correspond to one or more of a potential diagnosis, a risk factor, or potential treatment.

13. The one or more storage media of claim 11, wherein the plurality of candidate medical items include one or more of medicines, medical treatments, or medical tests.

14. The one or more storage media of claim 11, wherein the instructions, when executed by the one or more computing devices, further cause:
identifying, based on a patient identifier of the patient, one or more medical items, each of which was previously prescribed to the patient;
wherein determining the plurality of candidate medical items by the ML recommendation model is also based on the one or more medical items.

15. The one or more storage media of claim 14, wherein the instructions, when executed by the one or more computing devices, further cause:
receiving, from a user, input that selects a particular candidate medical item in the subset;
based on the input, updating an entry, in an order database, that corresponds to the patient to include a reference to the particular candidate medical item.

16. The one or more storage media of claim 11, wherein the instructions, when executed by the one or more computing devices, further cause:
receiving, from a user, input that indicates that one or more candidate medical items in the subset are incorrect recommendations;
updating the ML reinforcement learning model based on the input.

17. The one or more storage media of claim 11, wherein the instructions, when executed by the one or more computing devices, further cause:
receiving, from a user, input that indicates one or more reasons why one or more candidate medical items in the subset are incorrect recommendations;
updating the ML language model based on the input.

18. The one or more storage media of claim 11, wherein the instructions, when executed by the one or more computing devices, further cause:
receiving, from a user, input that indicates one or more reasons why one or more candidate medical items in the subset are incorrect recommendations;
updating the summary note data based on the input to generate an updated summary note data;
generating, by the ML language model, based on the updated summary note data, a second set of feature values;
generating, by the ML recommendation model, based on the profile of the patient, the profile of the physician, and the second set of feature values, a second plurality of candidate medical items;
generating, by the ML reinforcement learning model, a particular ranking of the second plurality of candidate medical items;
causing a subset of the second plurality of candidate medical items to be presented on the screen of the computing device based on the particular ranking.

19. The one or more storage media of claim 11, wherein:
receiving the summary note data is performed by a prompt engine;
the instructions, when executed by the one or more computing devices, further cause:
generating a set of instructions, and
causing the ML language model to access the set of instructions and the summary note data;
generating the set of feature values comprises generating the set of feature values also based on the set of instructions.

20. The one or more storage media of claim 19, wherein the instructions, when executed by the one or more computing devices, further cause:
prior to the ML recommendation model generating the plurality of candidate medical items, determining, by the prompt engine, whether the set of feature values satisfy one or more format criteria.
